(19) United States
(12) Patent Application Publication
(10) Pub. No.: US 2023/0144618 A1
(43) Pub. Date: May 11, 2023

(54) POWER SUPPLY DEVICE AND VITAL SENSOR

(71) Applicant: NIHON KOHDEN CORPORATION, Tokyo (JP)

(72) Inventors: Satoru TOGO, Tokorozawa-shi, Saitama (JP); Shinya OKUNO, Tokorozawa-shi, Saitama (JP); Toshiki AOKI, Tokorozawa-shi, Saitama (JP)

(21) Appl. No.: 17/906,872

(22) PCT Filed: Mar. 12, 2021
(86) PCT No.: PCT/JP2021/010157

§ 371 Date: Sep. 21, 2022

(30) Foreign Application Priority Data
Mar. 27, 2020 (JP) 2020-058255

(51) Int. Cl. H05K 9/00; A61B 5/1455; A61B 5/08; A61B 5/00

(52) U.S. Cl. CPC H05K 9/0024; A61B 5/14551; A61B 5/082; A61B 5/6826; A61B 5/682; H02J 2207/50; H02J 7/345

(57) ABSTRACT

A power supply device includes: a power supply circuit configured to supply power to a vital sensor; a capacitor electrically connected to the power supply circuit; a connector configured to supply power for charging the capacitor; and a shield case covering at least the power supply circuit and the capacitor to shield an electromagnetic wave.

### TECHNICAL FIELD

The presently disclosed subject matter relates to a power supply device configured to supply power for operating a vital sensor. The presently disclosed subject matter also relates to a vital sensor comprising the power supply device.

### BACKGROUND

U.S. Pat. No. 8,294,588 B2 discloses an example of a power supply device configured to supply power for operating a vital sensor adapted to be attached to a body of a subject to acquire a vital sign of the subject. The power supply device is configured such that a rechargeable battery is detachable from a sensor housing. The sensor housing has an electromagnetic wave shielding function with respect to an internal circuit, but no electromagnetic wave shield is provided in a location to which the rechargeable battery is attached.

### SUMMARY

### Technical Problem

It is demanded to enhance the convenience of the power supply device configured to supply power for operating the vital sensor.

### Solution to Problem

In order to meet the above demand, a first illustrative aspect of the presently disclosed subject matter provides a power supply device, comprising:

a power supply circuit configured to supply power to a vital sensor;

a capacitor electrically connected to the power supply circuit;

a connector configured to supply power for charging the capacitor; and

a shield case covering at least the power supply circuit and the capacitor to shield an electromagnetic wave.

In order to meet the above demand, a second illustrative aspect of the presently disclosed subject matter provides a vital sensor, comprising:

a probe adapted to be attached to a body of a subject, and configured to output a signal corresponding to a vital sign of the subject;

a power supply circuit configured to supply power to at least the probe;

a capacitor electrically connected to the power supply circuit;

a connector configured to supply power for charging the capacitor; and

a shield case covering at least the power supply circuit and the capacitor to shield an electromagnetic wave.

Since the capacitor is less degraded in power storage performance due to charging and discharging as compared with a rechargeable battery, it is not necessary to assume replacement. Accordingly, if a charging path from the external power source can be secured through the connector, the power supply circuit and the capacitor can be permanently accommodated in the shield case with a configuration having a higher sealing property against the electromagnetic wave. As a result, the convenience of the power supply device can be enhanced. For example, since the shielding property against the electromagnetic wave is enhanced, the vital sensor can be used even in an environment with strong electromagnetic noise such as during a magnetic resonance imaging (MRI) examination.

### BRIEF DESCRIPTION OF DRAWINGS

FIG. 1 illustrates an appearance of a vital sensor according to one embodiment.

FIG. 2 illustrates an example of a probe in the vital sensor of FIG. 1.

FIG. 3 illustrates another example of a probe in the vital sensor of FIG. 1.

FIG. 4 illustrates a functional configuration of a power supply device in the vital sensor of FIG. 1.

FIG. 5 illustrates another example of the configuration of the power supply device.

FIG. 6 illustrates another example of the configuration of the power supply device.

FIG. 7 illustrates an arrangement of a cylindrical lithium ion capacitor.

FIG. 8 illustrates an arrangement of the cylindrical lithium ion capacitor.

FIG. 9 illustrates an arrangement of a box-shaped lithium ion capacitor.

FIG. 10 illustrates an arrangement of the box-shaped lithium ion capacitor.

### DESCRIPTION OF EMBODIMENTS

Examples of embodiments will be described in detail below with reference to the accompanying drawings. In the drawings, the scale is appropriately changed in order to make each element to be described have a recognizable size.

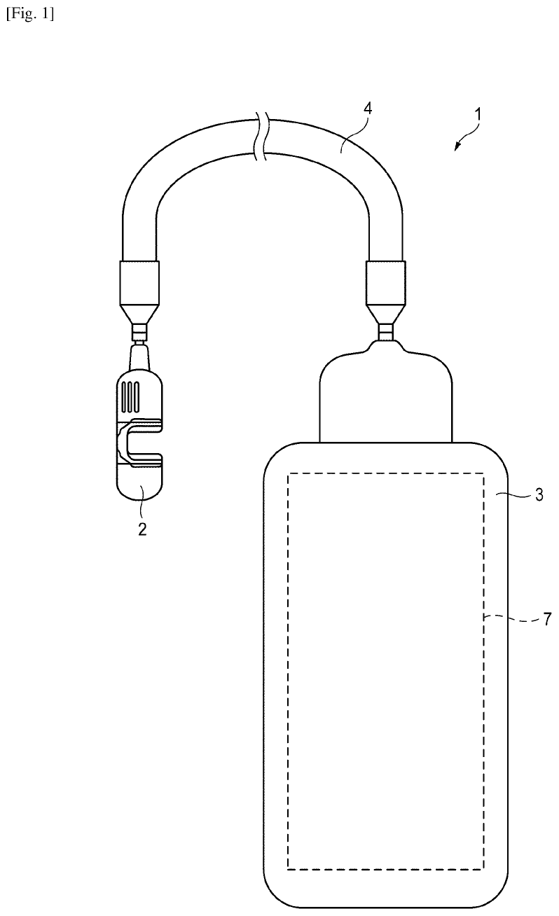

FIG. 1 illustrates an appearance of a vital sensor 1 according to an embodiment. The vital sensor 1 includes a probe 2, a casing 3, and a cable 4.

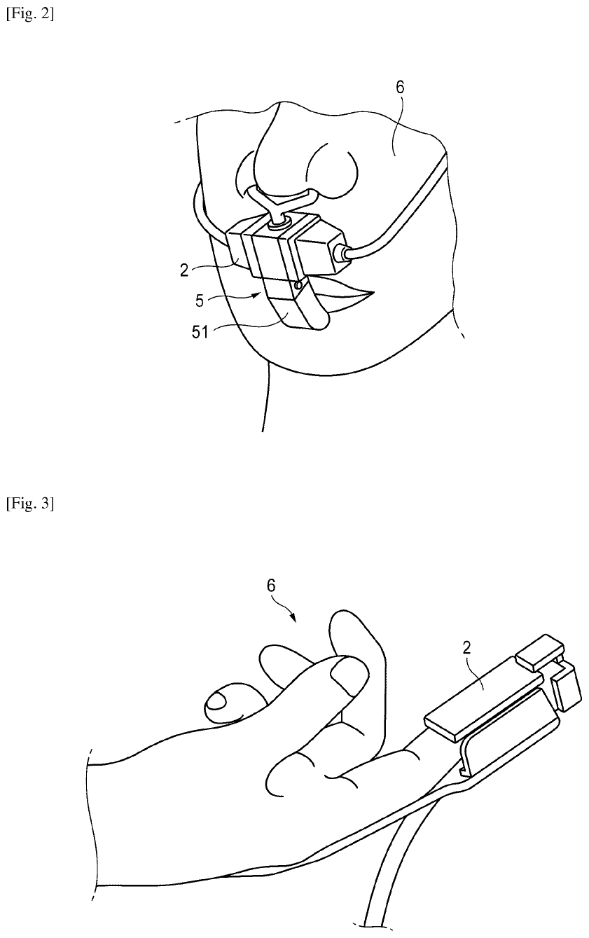

The probe 2 is configured to output a signal corresponding to carbon dioxide concentration of a subject. Specifically, the probe 2 includes a light emitting element and a light detecting element. The probe 2 may be attached to an adaptor 5 as illustrated in FIG. 2. The adaptor 5 is attached to a face of a subject 6. The adaptor 5 includes a passage 51 through which expired air of the subject 6 passes. The probe 2 is disposed such that the light emitting element and the light detecting element face each other with the passage 51 therebetween. The light emitted from the light emitting element is absorbed by the carbon dioxide contained in the expired air of the subject 6 when the light passes through the passage 51. Accordingly, the intensity of the light detected by the light detecting element changes in accordance with the carbon dioxide concentration. The carbon dioxide concentration is an example of the vital sign. The face is an example of the body of the subject.

In place of the probe 2 illustrated in FIG. 1, a probe 2 illustrated in FIG. 3 may be used. The probe 2 is configured to output a signal corresponding to transcutaneous arterial oxygen saturation (SpO2) of the subject 6. The probe 2 includes a light emitting element and a light detecting element. The probe 2 is attached to a fingertip of the subject 6. The light emitted from the light emitting element passes through a tissue of the fingertip, and is incident on the light detecting element. The incident intensity in the light detecting element is changed in accordance with oxyhemoglobin concentration contained in the arterial blood of the subject 6. The SpO2 corresponds to the oxyhemoglobin concentration. The SpO2 is an example of the vital sign. The fingertip is an example of the body of the subject.

As used herein with reference to the probe 2, the expression “adapted to be attached to a body of a subject” is meant to include a case where the probe is attached to the body of the subject indirectly via an adaptor as illustrated in FIG. 2, as well as a case where the probe is attached to the body of the subject directly as illustrated in FIG. 3.

As illustrated in FIG. 1, the casing 3 accommodates a power supply device 7. The power supply device 7 is configured to supply power for operating the vital sensor 1. The cable 4 electrically connects the probe 2 and the power supply device 7. Accordingly, the electric power is partially supplied to the probe 2 through the cable 4, whereby the above-described light emitting element or the like is operated.

Although not illustrated, the casing 3 accommodates a signal processor. The signal processor is configured to process the signal outputted from the probe 2, and transmit the processed signal to an external device such as a vital monitor device. The signal processor includes an adequate signal conversion circuit, a microcontroller, a communication circuit, and the like. The signal transmission to the external device may be performed through wired communication or wireless communication.

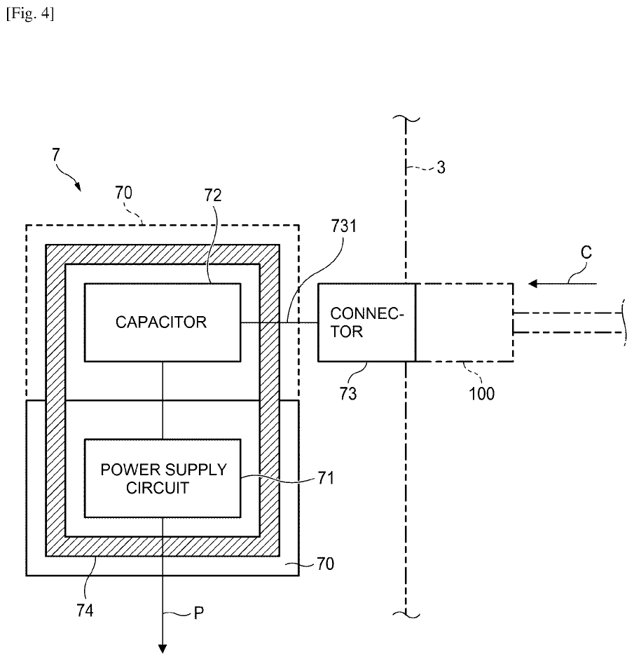

FIG. 4 illustrates a functional configuration of the power supply device 7. The power supply device 7 includes a substrate 70, a power supply circuit 71, a capacitor 72, a connector 73, and a shield case 74.

The capacitor 72 is an electronic component that can be charged and discharged. The capacitor 72 is electrically connected to the power supply circuit 71 supported by the substrate 70. Electrical energy stored in the capacitor 72 is supplied to the power supply circuit 71. The power supply circuit 71 performs appropriate processing on the electric energy, and outputs the electric energy as electric power P that causes each portion of the vital sensor 1 to operate.

The connector 73 is electrically connected to the capacitor 72 via a charging line 731. The connector 73 is disposed so as to be exposed on an outer face of the casing 3. When a connector 100 that has been connected to an external power source is connected to the connector 73, electric power C from the external power source is supplied to the capacitor 72 through the charging line 731. As a result, the capacitor 72 is charged.

The shield case 74 covers the power supply circuit 71 and the capacitor 72, thereby having a configuration capable of protecting the power supply circuit 71 and the capacitor 72 from the electromagnetic wave coming from the outside.

Since the capacitor is less degraded in power storage performance due to charging and discharging as compared with a rechargeable battery, it is not necessary to assume replacement. Accordingly, if a charging path from the external power source can be secured through the connector 73, the power supply circuit 71 and the capacitor 72 can be permanently accommodated in the shield case 74 with a configuration having a higher sealing property against the electromagnetic wave. As a result, the convenience of the power supply device 7 can be enhanced. For example, since the shielding property against the electromagnetic wave is enhanced, the vital sensor 1 can be used even in an environment with strong electromagnetic noise such as during a magnetic resonance imaging (MRI) examination.

In a case where the shield case 74 is supposed to be used in such an environment with strong electromagnetic noise, it is preferable to form the shield case 74 with a non-magnetic metal. Examples of the non-magnetic metal include nickel silver (an alloy of copper, zinc, and nickel), copper, aluminum, and stainless steel.

In FIG. 4, only the power supply circuit 71 is supported by the substrate 70. However, as illustrated by the dashed lines in the drawing, the capacitor 72 may also be supported by the substrate 70.

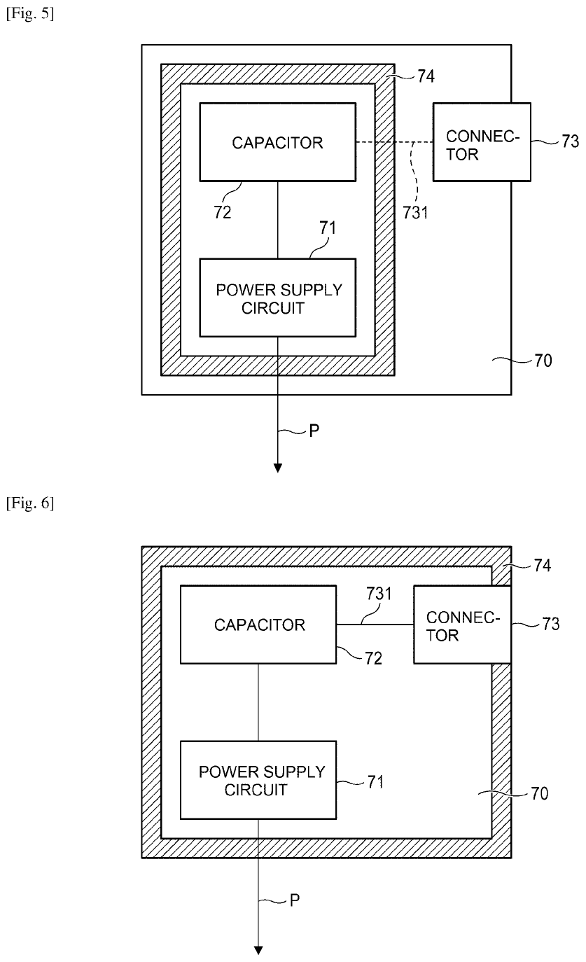

Alternatively, as illustrated in FIG. 5, in addition to the capacitor 72, the connector 73 may be supported by the substrate 70. In this case, the charging line 731 electrically connecting the connector 73 and the capacitor 72 may be formed in an inner layer of the substrate 70.

According to such a configuration, even when the connector 73 is disposed outside the shield case 74, the charging line 731 can be prevented from being directly exposed to the electromagnetic wave. Accordingly, the shielding property of the whole vital sensor 1 against the electromagnetic wave can be further enhanced.

By causing a portion of the substrate 70 to have the same potential as the shield case 74, the shielding property against the electromagnetic wave can be further enhanced.

Alternatively, as illustrated in FIG. 6, in addition to the power supply circuit 71 and the capacitor 72, the connector 73 may be configured to be accommodated in the shield case 74. In this case, in order to obtain the same shielding property as the configuration illustrated in FIG. 4, it is not necessary to form the charge line 731 in the inner layer of the substrate 70. Accordingly, an increase in the manufacturing cost of the substrate 70 can be suppressed.

In the configuration illustrated in FIG. 6, the capacitor 72 may be supported by the substrate 70, or may be disposed in the shield case 74 without being supported by the substrate 70.

In the present embodiment, a capacitor that performs charging and discharging with an electric double-layer phenomenon is used as the capacitor 72. Such a capacitor has a remarkably higher energy density than a so-called conventional capacitor, the increase in size of the power supply device 7 can be suppressed.

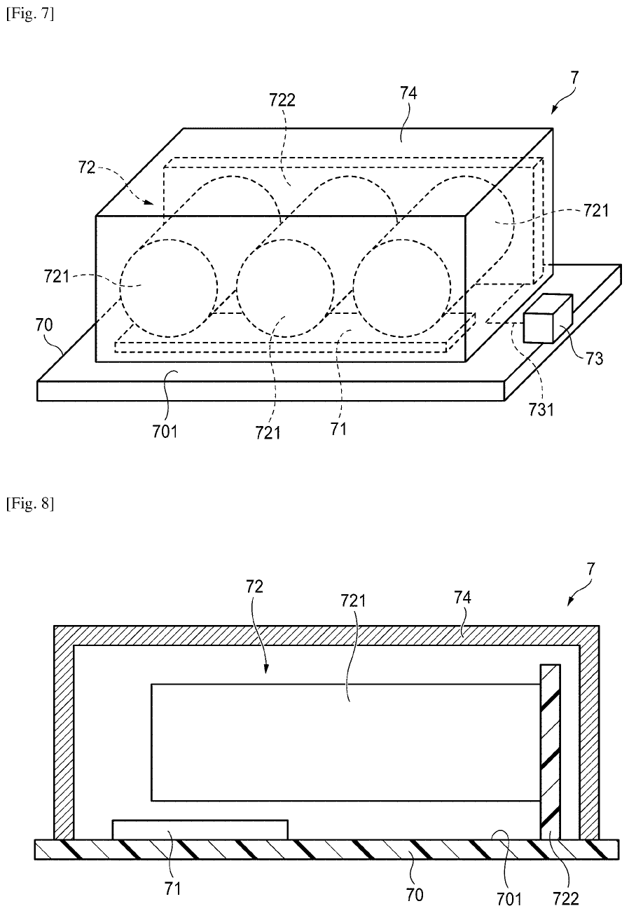

More specifically, a cylindrical lithium ion capacitor illustrated in FIGS. 7 and 8 is covered by the shield case 74 as the capacitor 72. The cylindrical lithium ion capacitor has a power storage portion 721 and a substrate portion 722. The power storage portion 721 has a cylindrical shape, and accommodates a plurality of electrode sheets and an electrolyte solution therein. The substrate portion 722 supports the power storage portion 721, and is provided with contacts for ensuring electrical connection with the power supply circuit 71 and the connector 73.

As illustrated in FIGS. 7 and 8, the substrate portion 722 is disposed such that the power storage portion 721 extends along a major face 701 of the substrate 70. In other words, the substrate portion 722 is disposed such that the axial direction of the cylindrical shape of the power storage portion 721 extends along the major face 701 of the substrate 70. As used herein, the term “a major face of a substrate” means a face having the largest area in a substrate.

According to such a configuration, the space utilization efficiency in the shield case 74 can be enhanced. In other words, the size of the power storage portion 721 can be increased within a range that can be tolerated in the accommodation space defined by the shield case 74. As a result, the energy density of the capacitor 72 can be increased.

The above embodiment is merely exemplary to facilitate understanding of the presently disclosed subject matter. The configuration according to each of the above embodiments can be appropriately modified without departing from the gist of the presently disclosed subject matter.

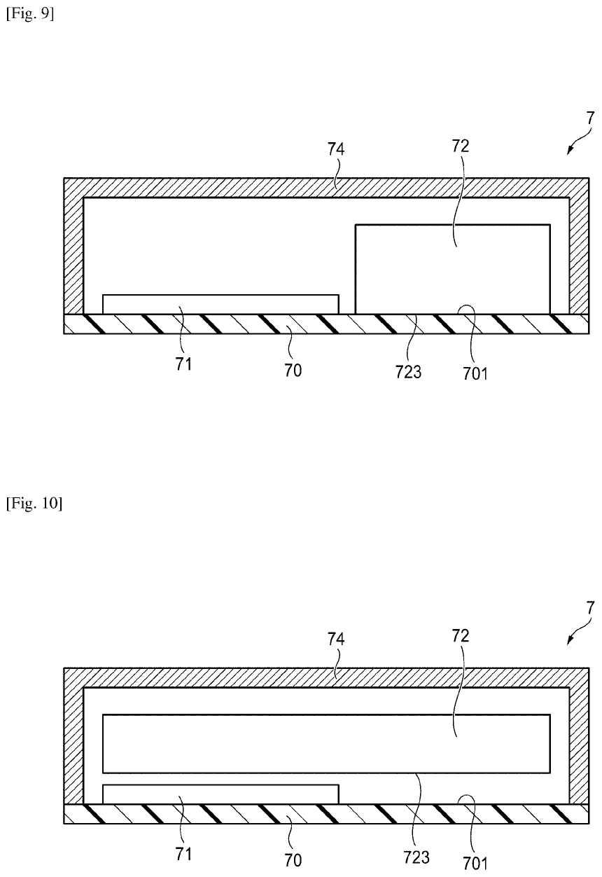

In the above embodiment, the cylindrical lithium ion capacitor is used as the capacitor 72. However, as illustrated in FIGS. 9 and 10, a lithium ion capacitor referred to as box-shaped or multilayered may be used as the capacitor 72.

Since this kind of lithium ion capacitor has a flat shape, a face 723 having the maximum area can be specified. As illustrated in FIGS. 9 and 10, by arranging the face 723 so as to face the major face 701 of the substrate 70, it is possible to enhance the utilization efficiency of the accommodation space defined by the shield case 74.

As illustrated in FIGS. 4, 5, 7, and 8, at least a portion of the shield case 74 need not be supported by the substrate 70. As illustrated in FIG. 6, the substrate 70 may be entirely disposed in the shield case 74.

The vital sign of the subject 6 that is acquired by the probe 2 is not limited to the carbon dioxide concentration in the respiratory air or the SpO2. According to the specification of the probe 2, pulse rate, blood pressure, oxygen concentration in respiratory air, light absorber concentration in arterial blood, and the like may be acquired.

The present application is based on Japanese Patent Application No. 2020-058255 filed on Mar. 27, 2020, the entire contents of which are hereby incorporated by reference.

1. A power supply device, comprising:
a power supply circuit configured to supply power to a vital sensor;
a capacitor electrically connected to the power supply circuit;
a connector configured to supply power for charging the capacitor; and
a shield case covering at least the power supply circuit and the capacitor to shield an electromagnetic wave.

2. The power supply device according to claim 1, wherein the capacitor is configured to perform charging and discharging with an electric double-layer phenomenon.

3. The power supply device according to claim 2, further comprising:
a substrate supporting at least the power supply circuit,
wherein the capacitor is a lithium ion capacitor including a power storage portion having a cylindrical shape and a substrate portion supporting the power storage portion, and
wherein the substrate portion is disposed such that the power storage portion extends along a major face of the substrate.

4. The power supply device according to claim 1, further comprising:
a substrate supporting at least the connector and the capacitor; and
a charging line formed in an inner layer of the substrate to connect the connector and the capacitor.

5. The power supply device according to claim 1, wherein the shield case is formed of a non-magnetic material.

6. A vital sensor, comprising:
a probe adapted to be attached to a body of a subject, and configured to output a signal corresponding to a vital sign of the subject;
a power supply circuit configured to supply power to at least the probe;
a capacitor electrically connected to the power supply circuit;
a connector configured to supply power for charging the capacitor; and
a shield case covering at least the power supply circuit and the capacitor to shield an electromagnetic wave.
